(19) United States
(12) Patent Application Publication
(10) Pub. No.: US 2018/0018624 A1
(43) Pub. Date: Jan. 18, 2018

(54) Method of Tracking and Tracing Syringes in the Pharmaceutical Industry

(71) Applicant: ZebraSci, Inc., Temecula, CA (US)

(72) Inventors: Hoang Thanh Nguyen, Riverside, CA (US); Jaan Noolandi, La Jolla, CA (US); Frederick Talley Gertz, Riverside, CA (US); Robert James Schultheis, Temecula, CA (US)

(21) Appl. No.: 15/430,351

(22) Filed: Feb. 10, 2017

Related U.S. Application Data
(60) Provisional application No. 62/362,444, filed on Jul. 14, 2016.

(51) Int. Cl. G06Q 10/08; A61J 1/18; G06F 19/00

(52) U.S. Cl. CPC G06Q 10/087; G06F 19/324; A61J 1/18; A61J 2205/10; A61J 2205/40

(57) ABSTRACT

A tracking and tracing method is provided during the life of the pharmaceutical container to improve the safety and efficacy of the pharmaceutical container and its content. An identification code is added to the surface of the pharmaceutical container, which is not visible under ambient light. The identification code contains encrypted information regarding temporal and physical properties of the pharmaceutical container and pharmaceutical fluid content. At multiple stages during the life of the pharmaceutical container the identification code is detected with an optical detection method. Given the material of the identification code, the identification code is only visible by using specific optical detection methods.

### CROSS-REFERENCE TO RELATED APPLICATIONS

This application claims priority from U.S. Provisional Patent Application 62/362,444 filed Jul. 14, 2016, which is incorporated herein by reference.

### FIELD OF THE INVENTION

This invention relates to methods and systems for tracking and tracing syringes in the pharmaceutical industry.

### BACKGROUND OF THE INVENTION

The FDA and other regulatory bodies want to track and trace of pharmaceutical products from the cradle to the grave. Current methods obscure views of the pharmaceutical product, damage the syringe (e.g., laser etching), or are expensive. Methods for tracking and tracing such as adding chemical tags on the outside of a syringe barrel require infrared (IR) or ultraviolet (UV) light to identify the tags, potentially damaging the drug inside the syringe. The present invention advances the art in providing a method to improve the efficacy and safety of a pharmaceutical product during its life.

### SUMMARY OF THE INVENTION

The present invention provides a method of safely tracking and tracing a pharmaceutical container content during the life of the pharmaceutical container, which improves safety and efficacy of the pharmaceutical fluid content of the pharmaceutical container during the life of the pharmaceutical container and its fluid content.

In the beginning of its life, a cylindrical pharmaceutical container is filled with a pharmaceutical content. An identification code is added to the surface of the pharmaceutical container. It is important that the material for the identification code is not visible under ambient light. The identification code contains encrypted information regarding temporal and physical properties of the pharmaceutical container and pharmaceutical fluid content of the pharmaceutical container. In one example, the identification code is defined by a pattern of dots or a bar code.

At multiple stages during the life of the pharmaceutical container the identification code is detected with an optical detection method. Given the material of the identification code, the identification code is only visible by using specific optical detection methods: (i) a diffused single-edge backlighting method in which only up to one half of the pharmaceutical container is illuminated and changes in the index of refraction according to changes of its light source angle of incidence are detected, or a (ii) a dark-field reflective method in which a collimated light source is used to illuminate the pharmaceutical container and light reflecting off the outer surface of the pharmaceutical container is detected according to changes of its light source angle of incidence.

The detection and decryption of the encrypted information of the identification code is carried out using a computer-implemented decrypting method. The decrypted identification code is verified at each stage of the multiple stages and outputs a safety and efficacy report pertaining to the pharmaceutical fluid content of the pharmaceutical container (the decryption steps utilize a computer-implemented method). Additionally, the method could include digitally subtracting background noise, which is obtained by the optical method of the surface that does not contain the identification code.

### BRIEF DESCRIPTION OF THE DRAWINGS

FIG. 1 shows a method according to an exemplary embodiment of the invention.

FIG. 2A shows a detected image according to an exemplary embodiment of the invention. The image was printed using a Xaar G6 print head from Engineered Printing Solutions (East Dorset VT) using a transparent ink solution.

FIG. 2B shows an enhanced image based on FIG. 2A applying image processing filters to improve contrast on the data matrix according to an exemplary embodiment of the invention.

FIG. 2C shows image thresholding based on FIG. 2B, applying image processing filters to create a binary image (necessary for data decryption) according to an exemplary embodiment of the invention.

FIG. 2D shows data decryption using a scanner based on FIGS. 2B-C. In this example, a mobile bar code scanner application was used to extract data. The meaning of the code 2WDY, DUU1 is merely exemplary. The code(s) could refer to any of, for example, lot # container, lot # of needle, needle and syringe dimensions, critical quality dimensions and variables, siliconization amount and pattern, code name for the drug. Companies in the industry could establish their own codes for various things. For example the code 2WDY, DUU1 could refer to the second batch produced at a filling site on a given week (W), a given day (D), and a given year (Y). The second part (DUU1) could refer to measured lubrication density (D), UU could refer to syringe volume, and whether it is plastic or glass, and the number 1 could refer to the needle size.

FIG. 3 shows according to an exemplary embodiment of the invention a schematic of diffused single-edge backlighting setup where the light is displaced by a distance D₁ from the center of the rotating specimen such that D₁ ranges from −R (standard diffused backlighting setup) to R (off-axis side lighting setup). R is dictated by the specimen dimensions, which may range from 0.15875 cm (e.g., inner diameter of a 1/16″ plastic tube) to over 35.56 cm (e.g., outer diameter of a 14″ barrel). Appropriate ranges for D₁ are thus −35.56 cm to 35.56 cm (as appropriate for the specimen). The light is placed a distance D₂ from the front surface of the light source to the outer diameter of the specimen where D₂ ranges from 0.0 cm (e.g., contacting the specimen) to 72 cm (e.g., the diameter of a 14″ barrel). The lens is placed a distance D₃ from the outer surface of the specimen such that D₃ ranges from 0.0 cm (e.g., contacting the specimen) to 80 cm (e.g., the focus distance for a very long working distance 1.0× magnification lens).

1=specimen (e.g. vial, syringe, cartridge, ampoule) rotated for line scan imaging,
2=diffused light source with one edge placed relative to specimen center,
3=line scan lens,
4=line scan imaging sensor,
R=radius of specimen,
D₁=displacement between edge of light source and center of specimen,
D₂=displacement between front of light source and outer diameter of specimen,
D₃=displacement between front of lens and outer diameter of specimen.

FIG. 4 shows according to an exemplary embodiment of the invention a schematic of standard diffused backlighting setup with the same distances D₂ and D₃ as defined in FIG. 3. FIG. 4 shows the standard way of illuminating an object. This method of FIG. 4 does not show the marks/dots shown in FIGS. 6A and 7A. When the barrel is illuminated by this standard method of FIG. 4, the marks/dots are invisible as shown in FIGS. 6B and 7B.

1=specimen (e.g. vial, syringe, cartridge, ampoule) rotated for line scan imaging,
2=diffused light source,
3=area scan lens,
4=area scan imaging sensor,
R=radius of specimen,
D₂=displacement between front of light source and outer diameter of specimen,
D₃=displacement between front of lens and outer diameter of specimen.

FIG. 5 shows according to an exemplary embodiment of the invention a schematic of reflected light setup such that the angle α between the light and the lens ranges from 0° (co-axial lighting) to 180° (low-angle lighting). The light is placed a distance D₄ from the specimen such that D₄ ranges from 0.0 cm (e.g., contacting the specimen) to as much as 100 m (e.g., a collimated laser light source). The lens is placed a distance D₃ from the outer surface of the specimen such that D₃ ranges from 0.0 cm (e.g., contacting the specimen) to 80 cm (e.g., the focus distance for a very long working distance 1.0× magnification lens).

1=specimen (e.g. vial, syringe, cartridge, ampoule) rotated for line scan imaging,
2=lens (to focus light),
3=imaging sensor (to record images),
4=collimated light source,
α=angle between light and lens,
D₄=displacement between front of lens and outer diameter of specimen,
D₅=displacement between front of light source and outer diameter of specimen,

FIG. 6A shows according to an exemplary embodiment of the invention a scan of dots with diffused single-edge backlighting at 0.75×,

FIG. 6B shows according to an exemplary embodiment of the invention a scan of dots with standard diffused backlighting at 0.75×.

FIG. 6C shows according to an exemplary embodiment of the invention a photo of syringe with inspection area highlighted. The syringe shown is a BD Hypak refillable glass syringe.

FIG. 7A shows according to an exemplary embodiment of the invention a sc′ of dots with reflected light with 0.75×,

FIG. 7B shows according to an exemplary embodiment of the invention a sc′ of dots with standard diffused backlighting at 0.75×.

FIG. 7C shows according to an exemplary embodiment of the invention a photo of BD Hypak refillable glass syringe with dots shown in FIG. 8A.

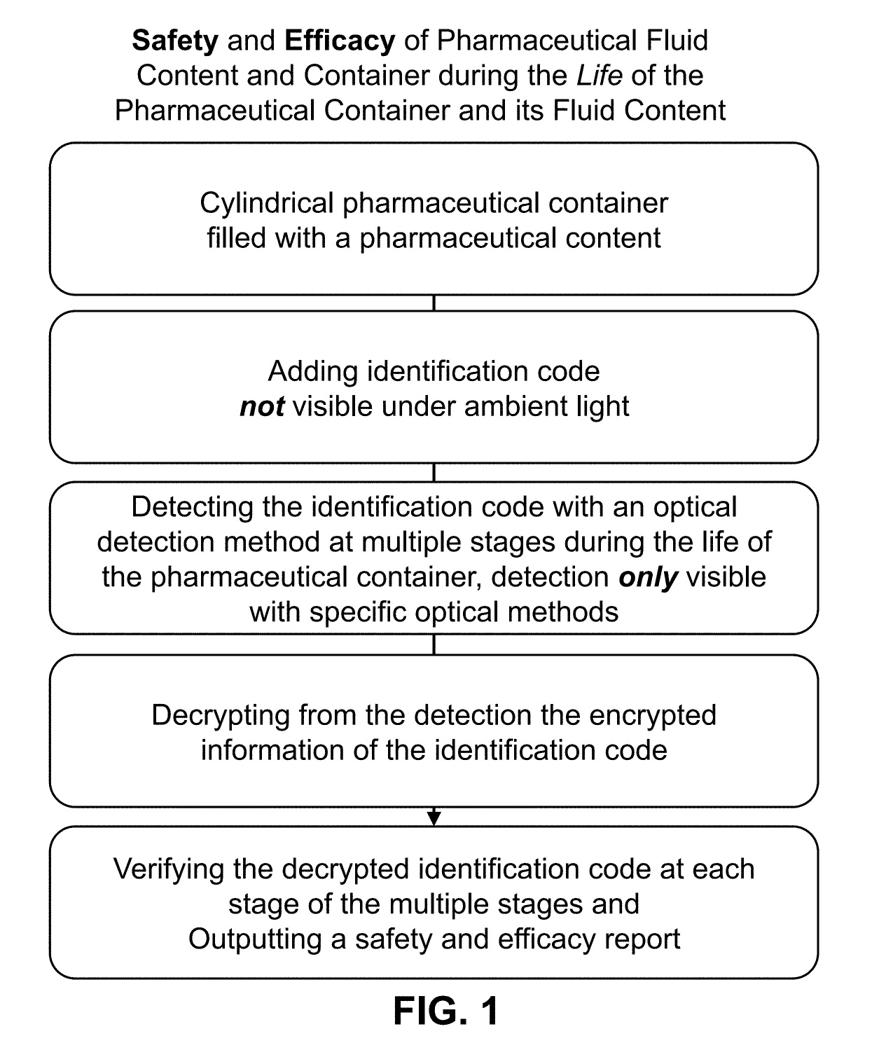

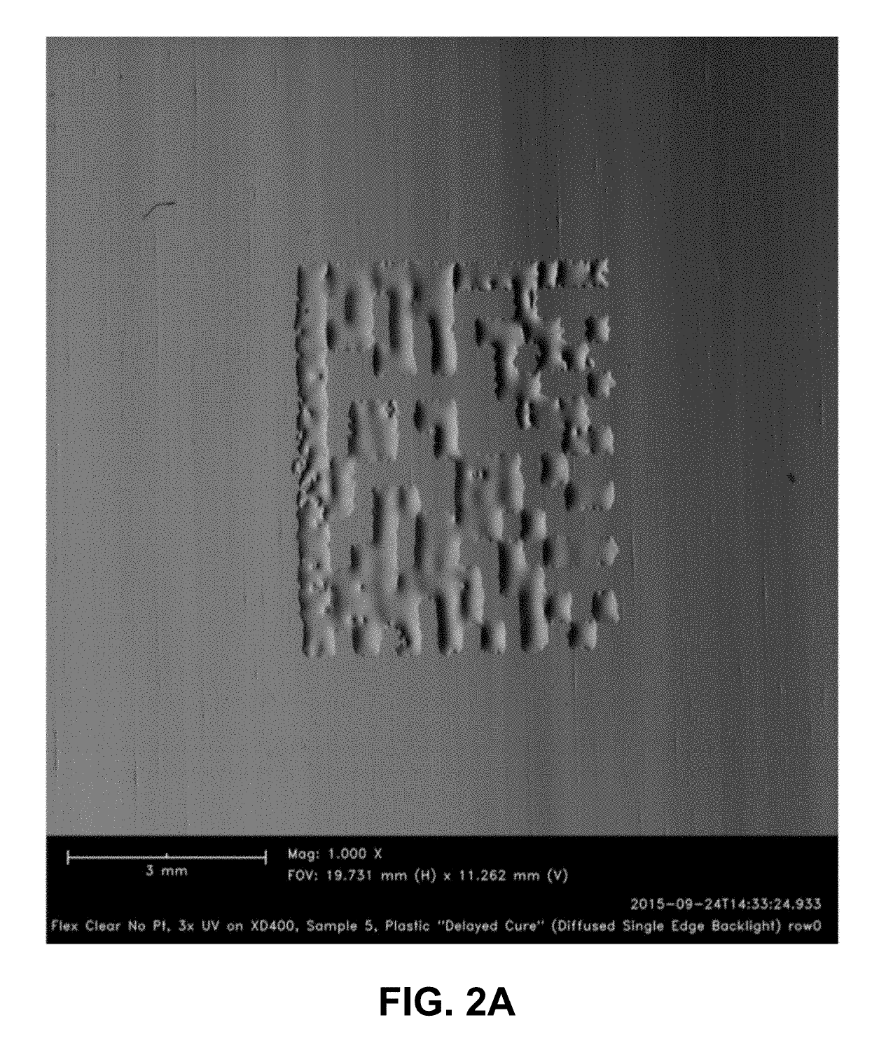

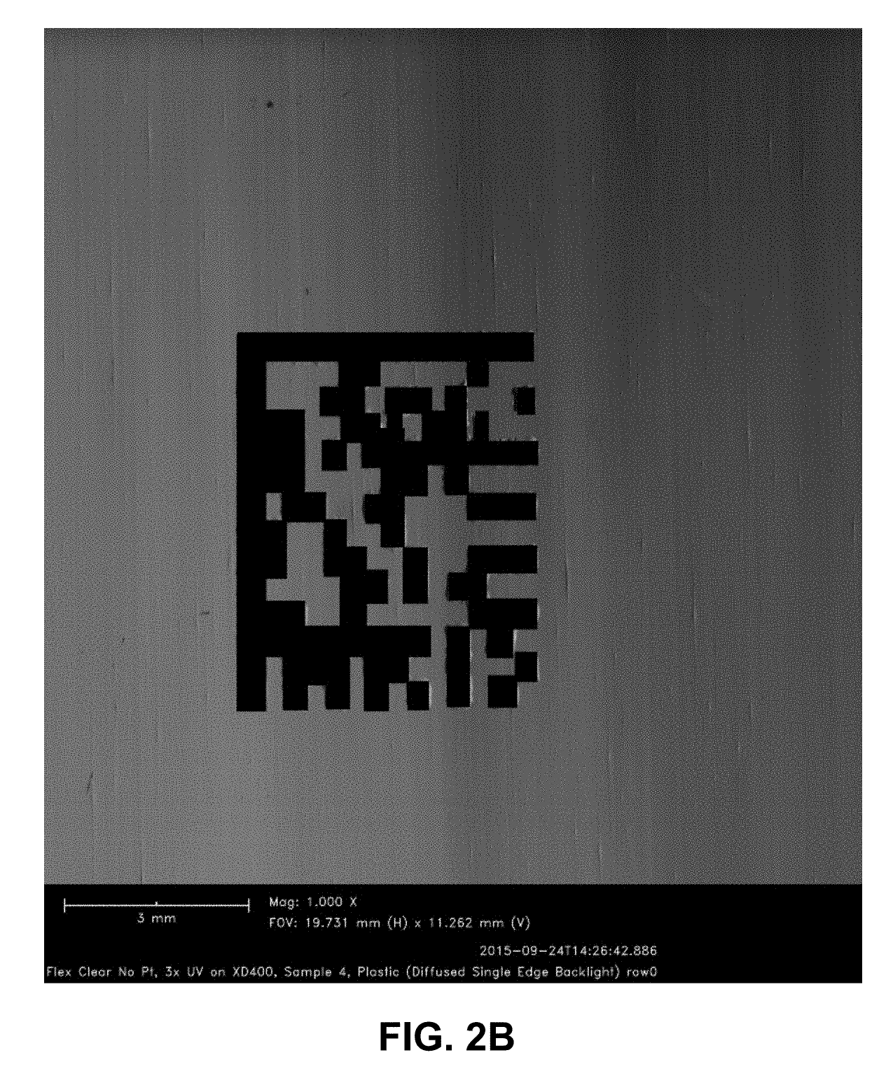

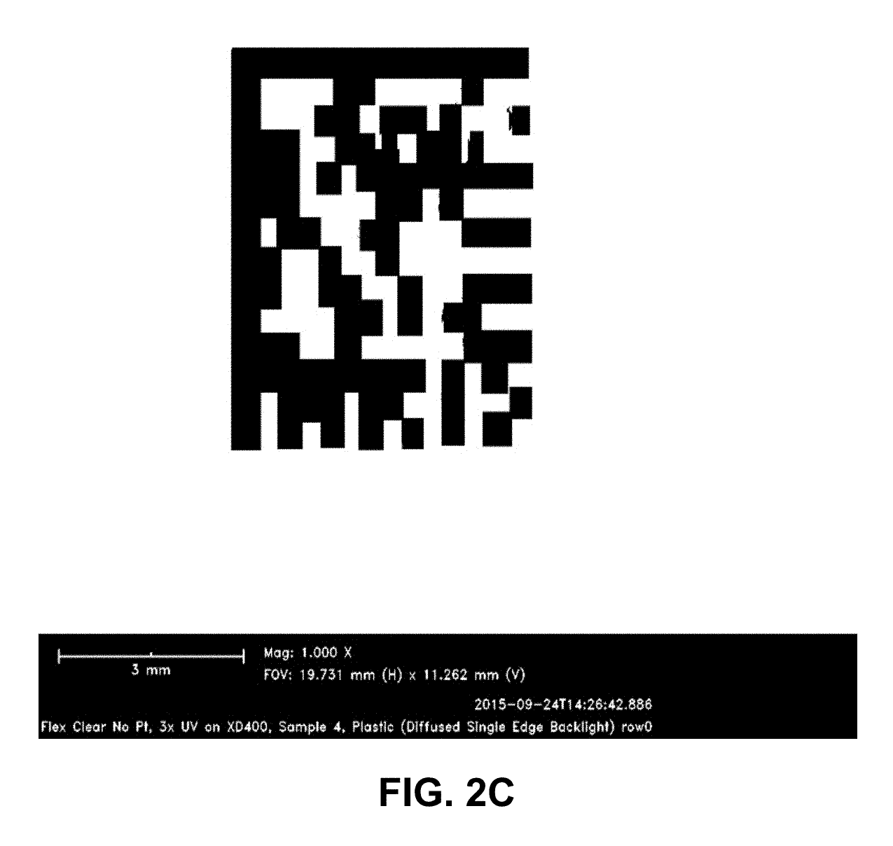

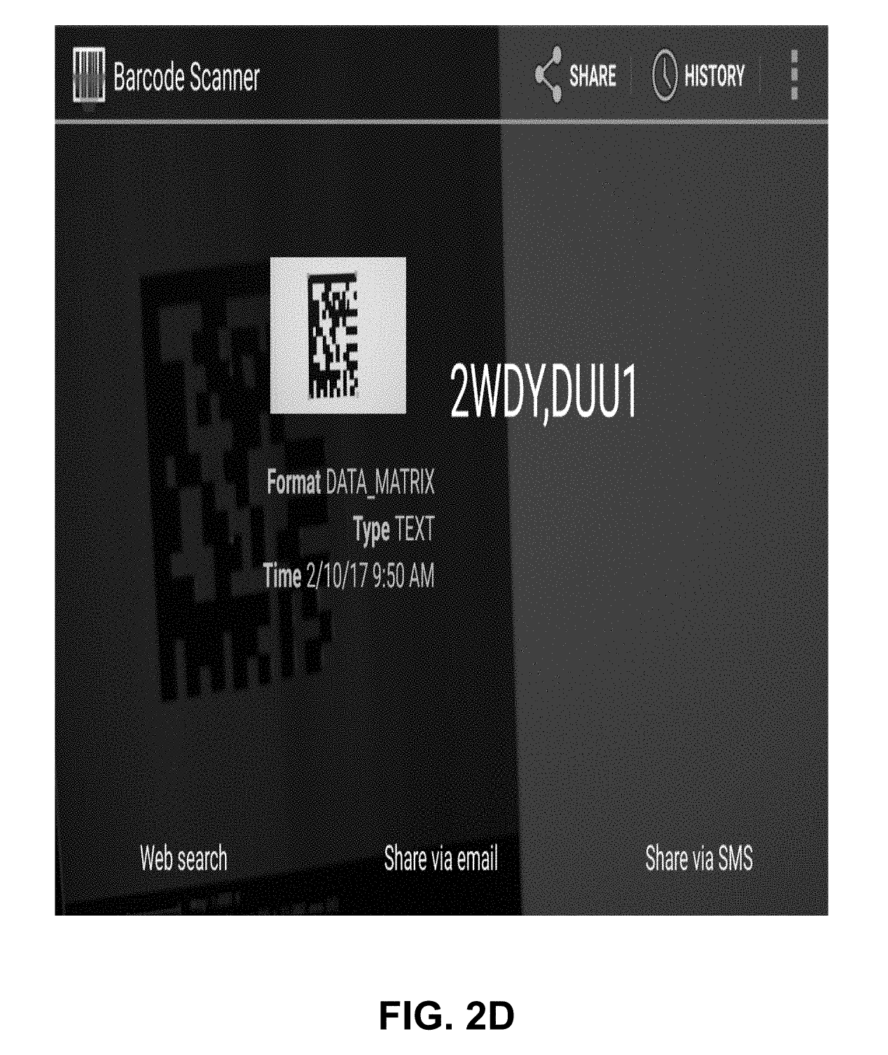

### DETAILED DESCRIPTION

The goal of the present invention is to put an identification (ID) (like a barcode, Morse code or 2D data matrix) of a clear material such as for example, but not limited to, cyanoacrylate or Krazy Glue) onto a pharmaceutical container. In one example, the ID could be a pattern of clear material dots where the number of dots, spacing of the dots and/or array formation of the dots defines the ID.

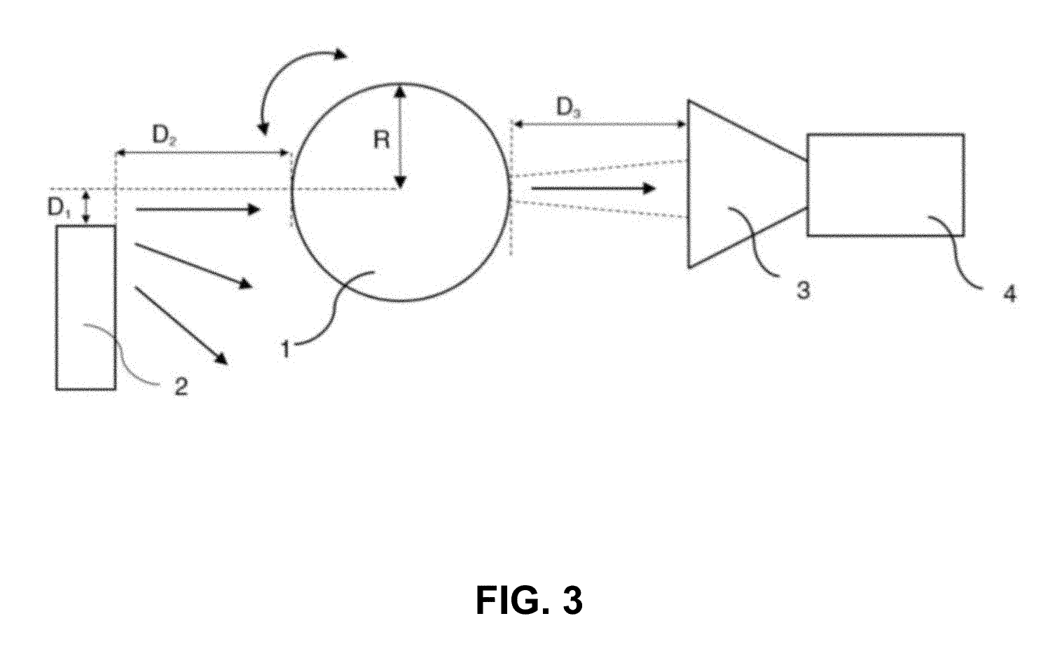

Since the identification (ID) is printed using a clear substance, not visible to ambient light, only an optical method that detects changes in the refractive index can detect the ID. In one such embodiment, the optical method as depicted in FIG. 3 or FIG. 5 can be used to detect this material.

The ID is used to track and trace the syringe through production, logging all quality information into a database. Examples of data that could be logged are:
lot # container,
lot # of needle,
needle and syringe dimensions,
critical quality dimensions and variables,
siliconization amount and pattern, etc.
code name for the drug.

This data is powerful as it would essentially be a Certificate of Analysis (CA) for each part produced. Pharmaceutical customers will find this useful as they can use the same ID to track the product through their processes. Most often, syringes are filled one time and are then labeled and packed elsewhere. Because of this, it is possible to mix up the product or to mislabel it. The ID can be used to ensure that the label will always match the product. Another advantage is that the ID can be used to orient the syringe in manufacturing when it is required. An example would be to orient the syringe needle correctly for the insertion of the needle shield.

In one embodiment, FIG. 3 shows a backlight is placed behind the specimen such that one half of the specimen is illuminated and one half of the specimen is not illuminated. A camera and lens is focused at the position where the light progresses from dark to light, only highlighting positions where there are changes in index of refraction. The specimen is rotated and imaged in fine increments as per the narrow field of view.

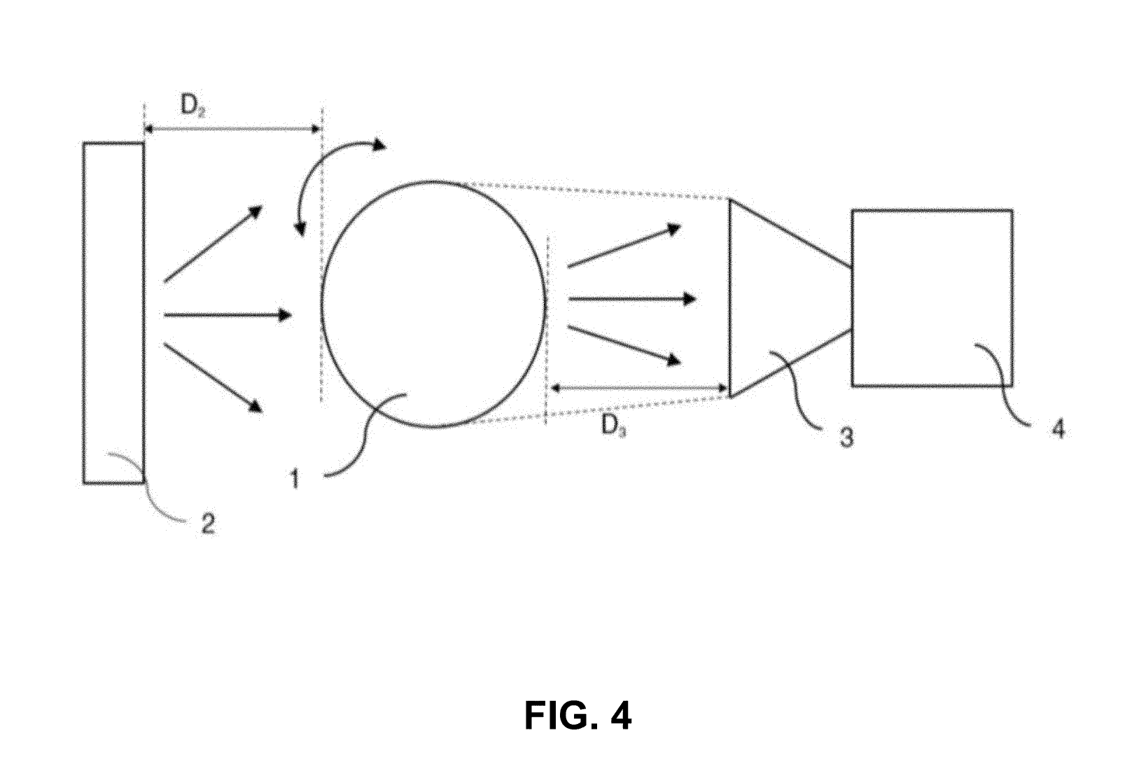

For comparison, FIG. 4 shows a backlight placed behind the specimen such that the entire specimen is illuminated. An area scan camera and lens is then focused on the specimen. Only opaque features such as defects or particles can be detected via light obscuration and will manifest themselves as dark silhouettes when captured by the camera. Clear material features will not be detected.

FIG. 5 shows a schematic of a setup according to an exemplary embodiment of the invention where a collimated light source is used to illuminate a rotating specimen and a camera setup is used to capture images of the light reflecting off of the first outer surface of the specimen. This produces very high-contrast images of any material on the specimen due to a change in index of refraction, for example a transparent droplet of a material other than glass.

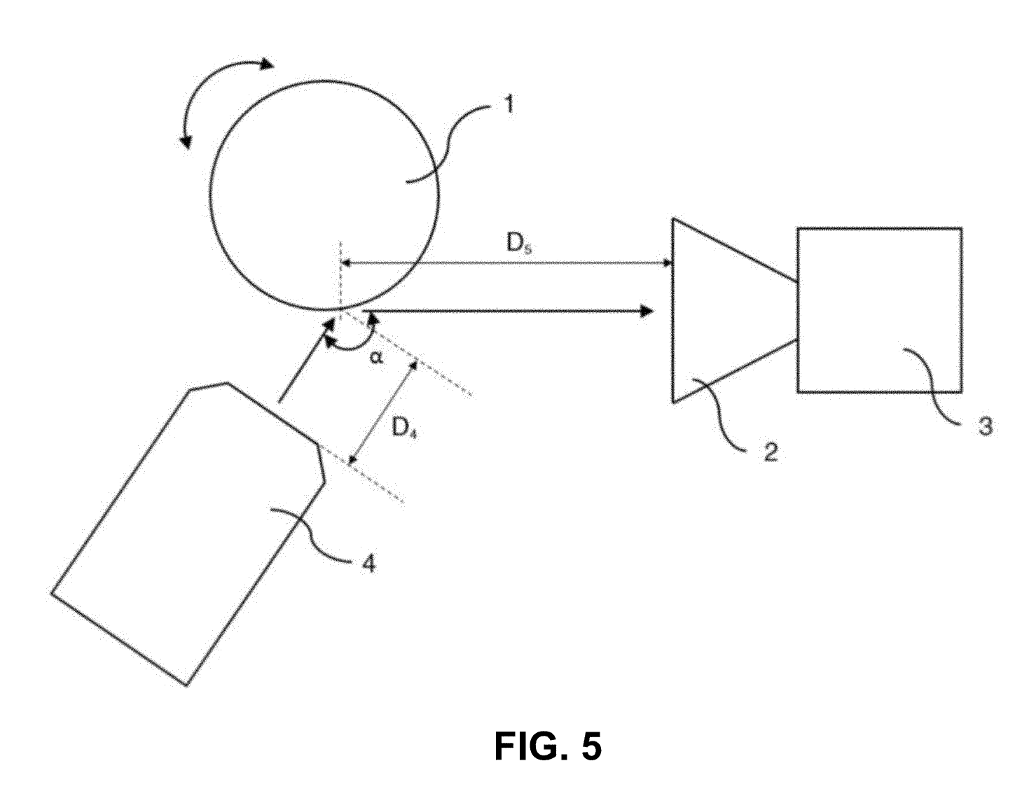

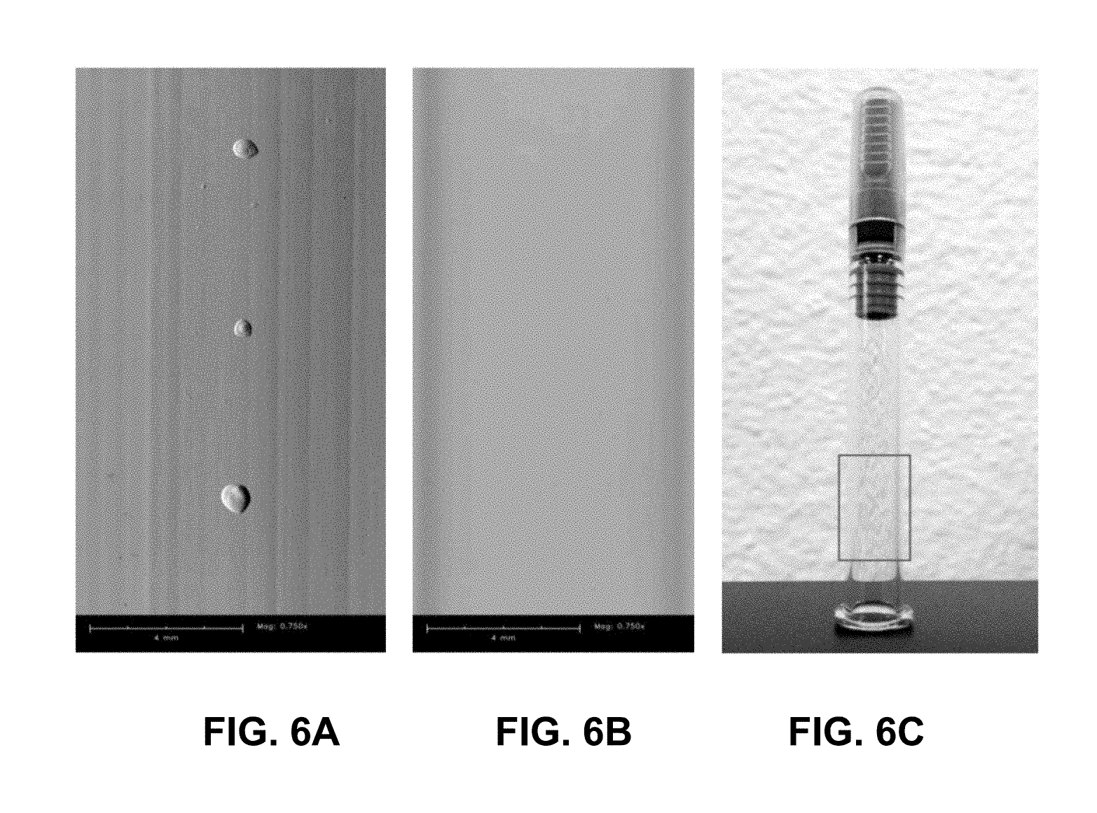

FIGS. 6A-B show the difference between a standard backlighting method (FIG. 6B) and the diffused single-edge backlighting (FIG. 3, FIG. 6A) to illuminate the same region (containing three dots) on the surface of the syringe.

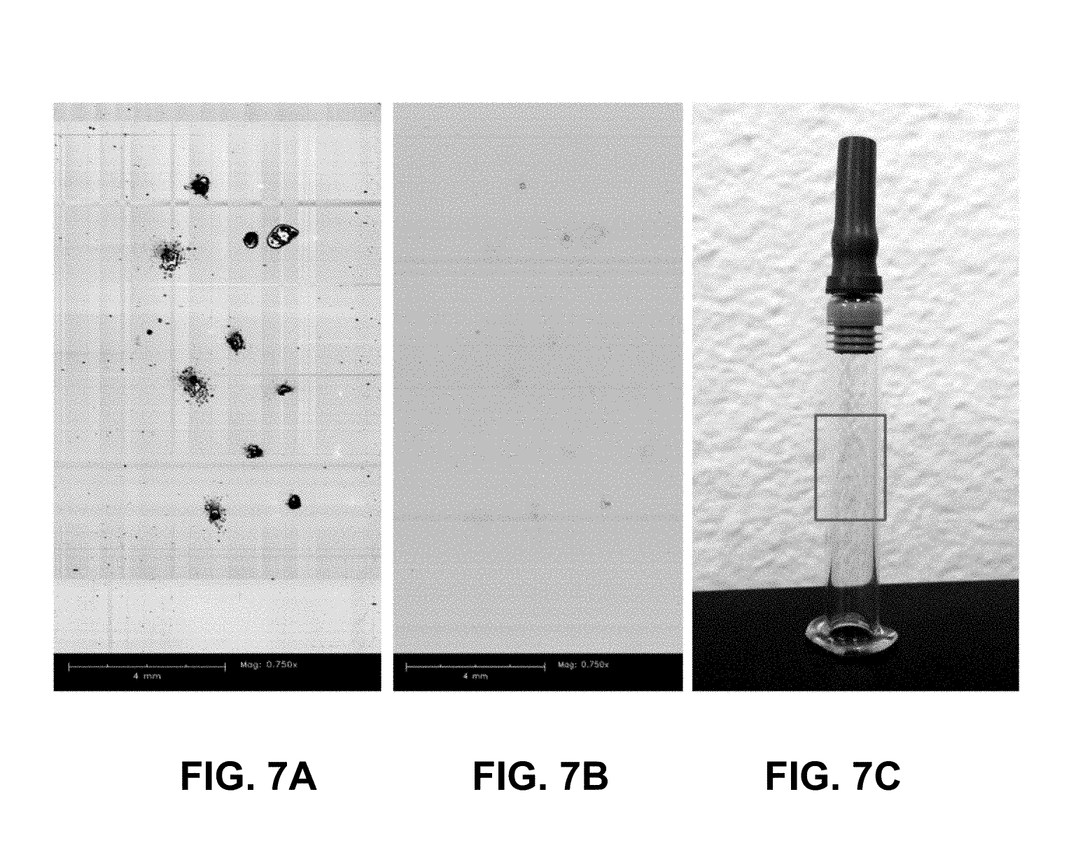

FIGS. 7A-C show the difference between a standard backlighting method (FIG. 7B) and the high definition light reflection method (FIG. 5, FIG. 7A) used to illuminate the three triangles of dots made of clear adhesive material (cyanoacrylate or Krazy Glue). FIG. 7B shows slight vestiges of the dots with standard diffuse lighting, but the ID cannot be identified.

In another embodiment of the invention a clear material (in this case without fluorescent tags) could be used to incorporate peptides with a given amino acid sequence which then could be recovered by swabbing and extracted for identification by electrospray ionization-mass spectrometry (ESI-MS) analysis via a simple liquid liquid extraction procedure.

What is claimed is:

1. A method of safely tracking and tracing a pharmaceutical container content during the life of the pharmaceutical container, comprising:
(a) providing a cylindrical pharmaceutical container filled with a pharmaceutical content;
(b) adding to the surface of the pharmaceutical container an identification code, wherein the identification code is made of a material that is not visible under ambient light, wherein the identification code contains encrypted information regarding temporal and physical properties of the pharmaceutical container and pharmaceutical fluid content of the pharmaceutical container;
(c) detecting the identification code with an optical detection method at multiple stages during the life of the pharmaceutical container, wherein the identification code is only visible by using the optical detection method comprising: (i) a diffused single-edge backlighting method in which only up to one half of the pharmaceutical container is illuminated and changes in index of refraction according to changes of its light source angle of incidence are detected, or a (ii) a dark-field reflective method in which a collimated light source is used to illuminate the pharmaceutical container and light reflecting off the outer surface of the pharmaceutical container is detected according to changes of its light source angle of incidence;
(d) decrypting from the detection the encrypted information of the identification code using a computer-implemented decrypting method; and
(e) verifying the decrypted identification code at each stage of the multiple stages and outputting a safety and efficacy report pertaining to the pharmaceutical fluid content of the pharmaceutical container, wherein the comparing and outputting utilizes a computer-implemented method,
wherein the method improves safety and efficacy of the pharmaceutical fluid content of the pharmaceutical container during the life of the pharmaceutical container and its fluid content.

2. The method as set forth in claim 1, wherein the method comprising digitally subtracting background noise which is obtained by the optical method of the surface that does not contain the identification code.

3. The method as set forth in claim 1, wherein the identification code is defined by a pattern of dots.
